(19) United States
(12) Patent Application Publication
(10) Pub. No.: US 2007/0000501 A1
(43) Pub. Date: Jan. 4, 2007

(54) Surgical procedure supplemental accessory controller and method utilizing turn-on and turn-off time delays

(76) Inventors: Lindsay T. Wert, Steamboat Springs, CO (US); Tim Refior, Englewood, CO (US)

Correspondence Address: JOHN R LEY, LLC, 5299 DTC BLVD, SUITE 610, GREENWOOD VILLAGE, CO, 80111, US

(21) Appl. No.: 11/173,199

(22) Filed: Jul. 1, 2005

(51) Int. Cl. A61B 19/00; A61B 18/14; A61B 18/20

(52) U.S. Cl. 128/898; 606/12; 606/32; 606/34

(57) ABSTRACT

A supplemental accessory used in a surgical procedure is energized in relation to activating a surgical unit used to perform the surgical procedure, by energizing the supplemental accessory after expiration of a predetermined turn-on time delay from the time when the surgical unit was activated. The supplemental accessory is energized after expiration of a basic fixed amount of time elapsing after the surgical unit has been activated, or after the surgical unit has been activated for a predetermined percentage less than all of the basic fixed amount of time, or after the surgical unit has been activated a predetermined multiple number of times during the basic fixed amount of time. The supplemental accessory is deenergized after the expiration of a predetermined turn-off time delay after the surgical unit was last deactivated or after the supplemental accessory was first energized, whichever occurs later.

This invention relates to energizing a supplemental accessory used in a surgical procedure in conjunction with activating a surgical unit which is used to perform the surgical procedure, such as for example, energizing a smoke evacuator in relation to activating an electrosurgical generator. More particularly, the present invention relates to a new and improved controller and method for energizing the supplemental accessory after a turn-on time delay relative to the activation of the surgical unit and for deenergizing the supplemental accessory after turn-off time delay relative to energizing the supplemental accessory or deactivating the surgical unit. Among other benefits, the initial effect of the surgical procedure may be better sensed by the surgeon and the number of intermittent operations of the supplemental accessory are minimized to reduce wear on the supplemental accessory.

### BACKGROUND OF THE INVENTION

Certain types of surgical units, such as electrosurgical generators and medical laser devices, apply energy to living tissue for the purpose of creating a surgical effect. For example, the energy may cut tissue, coagulate blood flow from tissue, cut and coagulate simultaneously, or restructure or resurface the tissue. The energy from an electrosurgical generator is high voltage electrical current applied at a radio frequency of approximately 400-700 kilohertz. The energy from a medical laser is light or photo energy. The electrical or light energy may be modulated to achieve a particular surgical effect.

The applied energy heats the tissue at the surgical site, creating smoke and odor, and sometimes creating a plume of airborne biological particles originating from the tissue at the surgical site. The smoke may obscure the surgical site and hinder the surgeon's ability to manipulate an applicator of the energy at the surgical site, which is sometimes referred to as a surgical accessory. The odor from heated tissue is unpleasant, and may become so pungent as to become nauseating and distracting from the procedure. There is considerable uncertainty about the health effects of airborne biological particles, particularly those particles from diseased tissues.

Smoke evacuators have been devised to eliminate the adverse effects of smoke, odor and airborne particle contaminants during surgery. A smoke evacuator draws air which contains the smoke, odor and airborne particle contaminants from the surgical site into a filtering and deodorizing device, and then exhausts the cleaned and deodorized air. The smoke no longer accumulates to the extent of obscuring the surgical site, the odor is eliminated, and airborne particles breathed by the operating room personnel are reduced.

To achieve these beneficial effects, smoke evacuators are typically energized and deenergized simultaneously with an electrosurgical generator or other surgical unit. The smoke evacuator is energized when the surgeon presses a finger switch on the handpiece-like energy applicator or when the surgeon steps on a foot switch to cause the surgical unit to deliver energy to the surgical site. Activation of the surgical unit in this manner is interpreted to energize the smoke evacuator and cause it to commence drawing air and contaminants from the surgical site. So long as the finger switch or the foot switch remains depressed, the surgical unit continues to deliver the energy to the surgical site and the smoke evacuator continues to draw the air and airborne contaminants from the surgical site. When the finger or foot switch is opened to terminate activation, the surgical unit and the smoke evacuator cease operation. Thus, the smoke evacuator is typically energized when the surgical unit is activated, and the smoke evacuator is typically deenergized when the surgical unit is deactivated.

A significant portion of a typical surgical procedure involves stopping bleeding from severed vessels. Surgical incisions invariably sever a few relatively larger vessels and a larger number of relatively smaller vessels. Bleeding from the larger vessels must be stopped first, because blood loss from the larger vessels represents a more serious condition. The surgical energy may be concentrated on the severed larger vessels over a somewhat-prolonged length of time to coagulate blood flow from the larger vessels, or sutures may be required to close the severed ends of the larger vessels. Once the bleeding from the larger vessels has been stopped, the lesser amount of blood oozing from the smaller vessels is stopped. Coagulating the smaller vessels is usually accomplished relatively quickly and easily by delivering short bursts of energy directly to the end of each severed smaller vessel. The surgeon rapidly moves from one small vessel to the next while quickly pressing and releasing the finger or foot switch to activate and deactivate the surgical unit as the applicator is brought into working distance of each small bleeding vessel. The surgical unit is switched on and off for short fractional-second applications of burst energy to create a spot-like coagulation effect at each small vessel. Typically, the surgeon moves quickly from one small bleeding vessel to another, spot-coagulating the small severed vessel by delivering the short energy bursts on an intermittent and rapidly-reoccurring basis.

During spot coagulation, and other circumstances where the surgical unit is intermittently and quickly activated and deactivated, the smoke evacuator is also energized and deenergized quickly on an equally rapid and intermittent basis. Rapidly and intermittently energizing and deenergizing the smoke evacuator can be relatively ineffective in evacuating the contaminants from the surgical site. Smoke, odor and airborne particles are not usually created immediately as a result of applying the energy to the tissue, because a finite amount of time is required before the energy heats the tissue enough to create the smoke, odor and airborne particle contaminants. Removing the contaminants also requires a finite amount of time to establish the air flow from the surgical site. The inertia of the still air resists the instantaneous removal of the contaminants from the surgical site. A fan or air pump within the smoke evacuator also requires a finite time to reach sufficient speed to become effective. Furthermore, energizing and deenergizing the smoke evacuator on a rapid intermittent basis causes noise, which may become a distraction to the surgeon and the operating room personnel. Energizing and deenergizing the smoke evacuator on a rapid and intermittent basis also accelerates the wear on the fan motor and other moving parts of the smoke evacuator, leading to accelerated wear and early failure.

One aspect of the problem associated with rapidly and intermittently energizing and deenergizing the smoke evacuator has been addressed by delaying the time when the smoke evacuator turns off or becomes deenergized, in relation to the time when the surgical unit was last activated. For example, smoke evacuators have remained energized after a time delay period of approximately two seconds after the time that the surgical unit was last activated. The turn-off delay allows the smoke, odor and airborne particles to continue to be removed as the tissue cools after the energy delivery ceases, because at least some smoke and odor will be created for a short time after the energy delivery terminates as a result of the residual heat in the tissue. If the surgical unit is activated within the turn-off time delay period, the smoke evacuator will continue to operate until the turn-off time delay has expired from the last activation of the surgical unit without it having been activated again.

The problems associated with rapidly and intermittently energizing and deenergizing the smoke evacuator also apply to other types of surgical accessories used with surgical units. For example, a lavage pump is sometimes activated in conjunction with an electrosurgical generator to deliver liquid to the surgical site or to remove liquid from the surgical site. Some types of medical lasers require gas flow to be delivered simultaneously with the light energy, to establish a particular type of gas medium between the light emitter and the tissue, or to cool certain functional components of the medical laser.

### SUMMARY OF THE INVENTION

The present invention recognizes that it is not necessary to instantly energize the smoke evacuator or other surgical procedure supplemental accessory simultaneously with activating the surgical unit. Instead, in many situations it is acceptable or desirable to delay energizing the smoke evacuator until after a relatively short turn-on time delay has elapsed following activation of the surgical unit. The short time delay allows the surgeon to sense the initial effect of the energy application on the tissue, to gauge the amount of energy delivered and to evaluate the response of the tissue to that energy, without the sensory distraction of immediately energizing the smoke evacuator or other supplemental accessory. Energizing the smoke evacuator or supplemental accessory simultaneously with activating the surgical unit has the potential of diminishing the best opportunity for the surgeon to sense these factors, because of the distraction of the added noise and air movement at the surgical site created by immediately energizing the smoke evacuator or supplemental accessory. The short turn-on time delay does not permit the smoke, odor and particulates to accumulate to the point where it is impossible to remove those contaminants once the smoke evacuator is energized after the short turn-on time delay. On the other hand, activation of the surgical unit continuously during the initial turn-on delay time, or for a predetermined significant portion of the initial turn-on time delay, or for a predetermined plurality of rapid intermittent activations during the initial turn-on time delay, indicates that it is desirable to shorten the initial turn-on time delay before energizing the smoke evacuator or other surgical unit. These conditions indicate that the contaminants may start rapidly accumulating and earlier activation of the smoke evacuator is desirable.

Once energized, the supplemental accessory remains energized for a predetermined turn-off time delay after the supplemental accessory was initially energized or the surgical unit was last deactivated. Activation of the surgical unit during the time while the supplemental accessory remains energized resets the turn-off time delay, and continues the energization of the supplemental accessory. Continuing the energization of the supplemental accessory avoids the added wear on the supplemental accessory created by repeatedly stopping and starting the operation of the supplemental accessory. A surgical procedure which progresses at a relatively continuous pace will therefore provide the opportunity to remove the smoke, odor and particulate contaminants on a relatively continuous basis throughout the surgical procedure.

In accordance with these aspects, the present invention relates to a method of energizing a supplemental accessory in relation to activating a surgical unit, and a controller for controlling the energizing of a supplemental accessory in relation to activation of a surgical unit. The method involves activating the surgical unit to apply energy and energizing the supplemental accessory after expiration of a predetermined turn-on time delay after the surgical unit has been activated. The controller executes a control algorithm which causes the supplemental accessory to be energized in relation to an activation signal asserted by the surgical unit upon delivering the energy. The control algorithm operatively closes an accessory switch to cause the supplemental accessory to be energized and opens the accessory switch to cause the supplemental accessory to be deenergized. The control algorithm causes the controller to operatively close the accessory switch after expiration of a predetermined turn-on time delay from the assertion of the activation signal. Preferably, the surgical unit is an electrosurgical generator or a surgical laser, and the supplemental accessory is a smoke evacuator, lavage pump or fluid pump.

Certain preferred aspects of the method involve establishing the predetermined turn-on time delay as a basic fixed amount of time elapsing after the surgical unit has been activated, establishing the predetermined turn-on time delay as an amount of time less than the basic fixed amount of time upon activating the surgical unit for a predetermined percentage less than all of the basic fixed amount of time, and energizing the supplemental accessory when a counted number of activations occurring during the basic fixed amount of time equals a predetermined count number greater than one. Other preferred aspects of the method involve deactivating the surgical unit to cease applying energy after the expiration of a predetermined turn-off time delay when the surgical unit was last deactivated or when the supplemental accessory was first energized, whichever occurs later. Preferably the predetermined turn-off time delay is greater than the predetermined turn-on time delay.

Certain preferred aspects of the control algorithm executed by the controller involve establishing the predetermined turn-on time delay as a basic fixed amount of time. The control algorithm preferably causes the controller to count the amount of time that the activation signal is asserted during the basic fixed amount of time and close the accessory switch before expiration of the predetermined turn-on time delay when the counted amount of time that the activation signal is asserted equals a predetermined percentage less than all of the basic fixed amount of time, or to count the number of assertions of the activation signal during the basic fixed amount of time and close the accessory switch before expiration of the predetermined turn-on time delay upon the counted number of assertions of the activation signal equaling a predetermined number greater than one. In addition the control algorithm preferably causes the controller to establish the predetermined turn-on time delay as the earliest one to occur of the expiration of the basic fixed amount of time after assertion of the activation signal, of the activation signal being asserted for a predetermined percentage less than all of the basic fixed amount of time, or of the activation signal being asserted a predetermined multiple number of times during the basic fixed amount of time. Preferably still, the control algorithm causes the controller to open the accessory switch after the expiration of a predetermined turn-off time delay after the last to occur of the assertion of the activation signal or the closure of the accessory switch.

A more complete appreciation of the scope of the present invention and the manner in which it achieves the above-noted and other improvements can be obtained by reference to the following detailed description of presently preferred embodiments taken in connection with the accompanying drawings, which are briefly summarized below, and by reference to the appended claims.

### BRIEF DESCRIPTION OF THE DRAWINGS

FIG. 1 is a block diagram illustrating a generalized application of the present invention to an electrosurgical generator surgical unit and a smoke evacuator supplemental accessory.

FIG. 2 is a flowchart illustrating the program flow and basic functionality of a control algorithm for controlling the energizing of a supplemental accessory in accordance with the present invention, executed by an accessory controller such as that shown in FIG. 1.

FIGS. 3, 4, 5, 6 and 7 are each interrelated pairs of state transition diagrams illustrating activation and deactivation states of the surgical unit as causing energization and deenergization states of the supplemental accessory, with the diagrams having some common timing interrelationships.

### DETAILED DESCRIPTION

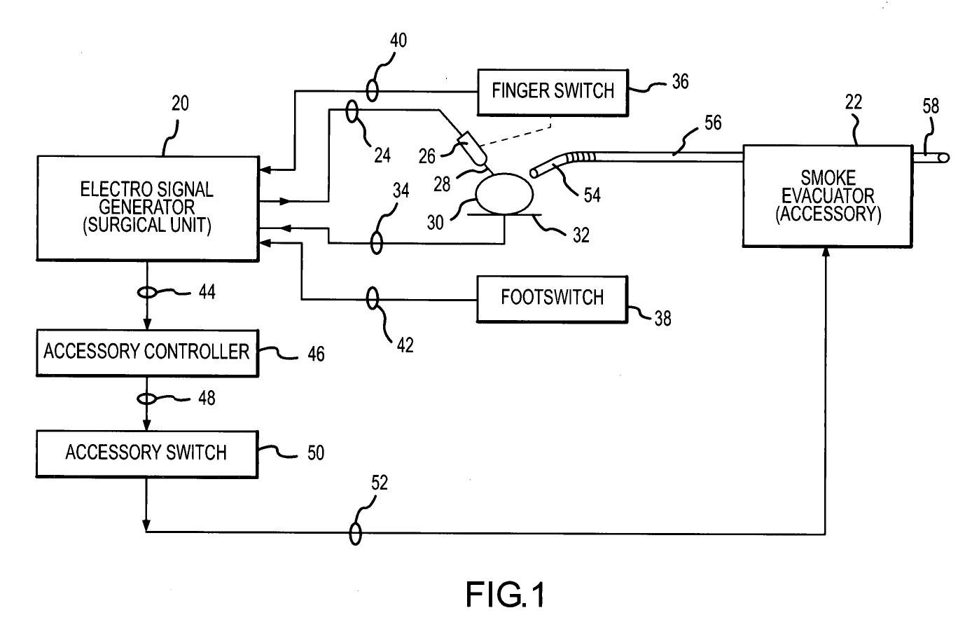

An application of the present invention is for energizing and deenergizing a supplemental accessory used in a surgical procedure with a surgical unit in relation to conditions associated with activation and deactivation of a surgical unit. As shown in FIG. 1, the surgical unit is represented by an electrosurgical generator 20, and the supplemental accessory is represented by a smoke evacuator 22. The electrosurgical generator 20 delivers high-voltage radio frequency (RF) current as an electrosurgical waveform 24 to a handpiece 26, which is sometimes referred to as a surgical accessory. The high-voltage RF current of the waveform 24 is applied from an active electrode 28 of the handpiece 26 to tissue of the patient 30 at a surgical site. The current flows through the patient 30 to a return electrode 32 which also contacts the patient. The current returns to the generator 20 at 34, thus completing an electrical circuit from the electrosurgical generator 20 through the patient 30. The high-voltage RF current of the electrosurgical waveform 24 interacts with the tissue at the surgical site to create a surgical effect, such as cutting, coagulation, or both.

The electrosurgical generator 20 is activated to deliver the high-voltage RF current in response to the closure of a typical finger switch 36 or the closure of a typical foot switch 38. The finger switch 36 is typically part of the handpiece 26, or is otherwise associated with the handpiece 26, so the surgeon can activate the electrosurgical generator 20 while holding the handpiece 26. The foot switch 38 is placed on the floor of the operating room so that the surgeon may step on it during the surgical procedure to activate the electrosurgical generator 20. Upon closing the finger switch 36 by pressing on it, an activation request signal 40 is sent to the electrosurgical generator 20. Upon closing the foot switch 38 by stepping on it, an activation request signal 42 is sent to the electrosurgical generator. The electrosurgical generator 20 responds to either activation request signal 40 or 42 by delivering the electrosurgical waveform 24 of high-voltage RF current. In some types of surgical units such as medical lasers, nothing comparable to the return electrode 32 is used, since the application of energy does not require a closed energy flow circuit through the patient.

The electrosurgical generator 20 also responds to the activation request signals 40 and 42 by delivering a generator activation signal 44, approximately simultaneously with delivering the electrosurgical waveform 24. The generator activation signal 44 is delivered in response to either activation request signal 40 or 42, after all of the internal safeguard and status checks have been performed by the electrosurgical generator 20, when the electrosurgical generator 20 delivers the electrosurgical waveform 24.

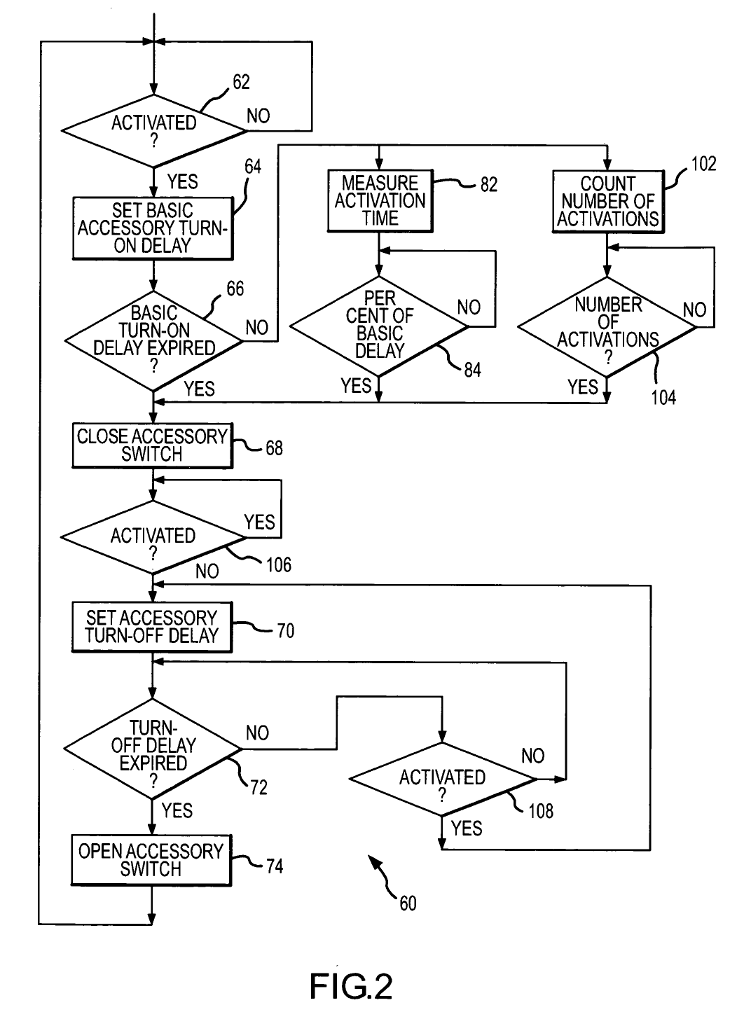

In the arrangement shown in FIG. 1, a supplemental accessory controller 46 responds to the generator activation signal 44. The accessory controller 46 preferably includes a processor, microcontroller, state machine, or other digital logic elements which execute a control algorithm 60 (FIG. 2) which causes the supplemental accessory to be energized in response to certain conditions associated with the assertion of the generator activation signal 44. An accessory control signal 48 is supplied by the accessory controller 46 in response to the execution of the control algorithm 60 (FIG. 2). An accessory switch 50 closes and opens in response to the assertion and de-assertion of the accessory control signal 48. Closure of the accessory switch 50 asserts an accessory energizing signal 52 to the smoke evacuator 22, causing the smoke evacuator 22 to commence operation. Opening the accessory switch 50 de-asserts the energizing signal 52, causing the smoke evacuator 22 to cease operation. In general, the accessory switch 50 conducts conventional electrical power to the smoke evacuator 22 or other supplemental accessory when closed, and the accessory switch 50 prevents conventional electrical power from energizing the smoke evacuator 22 when opened.

The smoke evacuator 22 responds to the energizing signal 52 by initiating operation of a conventional internal fan or air pump (neither shown) which commences evacuating the air which carries smoke, odor and particle contaminants from the surgical site at the active electrode 28. A conventional evacuation wand 54 collects the air containing the smoke, odor and particle contaminants from the surgical site, and an evacuation hose 56 which is connected to the wand 54 conveys these contaminants to the smoke evacuator 22. The contaminants are removed by conventional filter and contaminant-neutralizing components (none shown) within the smoke evacuator 22. Cleaned, deodorized and neutralized air is discharged from the smoke evacuator 22 through a discharge port 58. The smoke evacuator 22 responds to the de-assertion of the energizing signal 52 by stopping the evacuation of air and contaminants from the surgical site and stopping the cleaning, deodorizing and neutralizing of the air flowing within the smoke evacuator 22. Thus, the smoke evacuator 22 is energized and removes the contaminants from the air obtained at the surgical site when the energization signal 52 is asserted, and the smoke evacuator is deenergized and stops operating when the energization signal 52 is de-asserted. The execution of the control algorithm 60 (FIG. 2) in response to the assertion and deassertion of the generator activation signal 44 determines the conditions under which the energization signal 52 is asserted and de-asserted.

In most cases, the electrosurgical generator 20 will have an internal processor, microcontroller, state machine, or other digital logic control elements which will execute the control algorithm 60 (FIG. 2) and deliver the accessory control signal 48 to the accessory switch 50. In this case, the accessory controller 46 will be a programmed part of the functionality of the electrosurgical generator. The accessory switch 50 will also typically be part of the electrosurgical generator. The accessory switch 50 is typically implemented as a relay which closes and opens in response to the assertion and de-assertion of the accessory control signal 48, to conduct conventional electrical power to the smoke evacuator 22 or other special accessory. However, it is within the scope of the present invention to provide a separate accessory controller 46 and accessory switch 50, apart from the electrosurgical generator 20 or other surgical unit, and execute the control algorithm 60 (FIG. 2) with an internal processor, microcontroller, state machine or other digital logic control elements of the separate accessory controller 46. In such circumstances, the electrosurgical generator 20 or other surgical unit need only assert and de-assert the generator activation signal 44 conjunctively with the delivery of the electrosurgical waveform 24.

The control algorithm 60 for energizing the supplemental accessory, shown in FIG. 2, commences execution in response to the assertion of the activation signal 44, which occurs from closing the finger switch 36 or the foot switch 38 to assert an activation request signal 40 or 42, respectively (FIG. 1). The control algorithm 60 determines at 62 whether the activation signal 44 (FIG. 1) has been asserted. An affirmative determination at 62 establishes a basic turn-on time delay at 64. After the turn-on time delay expires, as determined at 66, the accessory switch (50, FIG. 1) is closed, as shown at 68. The basic turn-on time delay may be shortened depending upon different circumstances of activating the surgical unit. Closing the accessory switch at 68 causes the smoke evacuator or other supplemental accessory to be energized. Once the accessory switch has been closed at 68 to energize the supplemental accessory, a turn-off time delay is established at 70 during which the smoke evacuator or other supplemental accessory remains energized. The amount of time that the supplemental accessory remains energized is measured from the time that the supplemental accessory is initially energized or from the time that the surgical unit is deactivated. Activating the surgical unit while the supplemental accessory remains energized resets the turn-off time delay. Once the turn-off time delay has expired as determined at 72, the accessory switch is opened at 74. Opening the accessory switch deenergizes the supplemental accessory.

Details of establishing and adjusting the amount of the turn-on time delay and of establishing the amount of turn-off time delay for energizing and deenergizing the supplemental accessory, relative to different activation and deactivation conditions of the surgical unit are illustrated in the process flow of the control algorithm 60. Until an activation signal is affirmatively determined at 62, a wait loop is established at 62 to wait for the assertion of the activation signal when the surgical unit becomes activated. Once activation is affirmatively determined at 62, the basic accessory turn-on time delay is established at 64. The basic turn-on time delay is preferably a selected amount of time, for example, one second. The amount of time for the basic turn-on time delay established at 64 should be long enough to accommodate the recognition of other activation conditions, described below, which are effective to shorten the basic turn-on time delay established at 64.

After the basic turn-on time delay has been set at 64, a determination is made at 66 as to whether the basic turn-on time delay has expired. Upon expiration of the turn-on time delay, as indicated by an affirmative determination at 66, the accessory switch is closed at 68. Closure of the accessory switch results in energizing the smoke evacuator or other supplemental accessory. Energizing the supplemental accessory therefore occurs in response to activating the surgical unit, as determined at 62, followed by establishing the basic turn-on time delay at 64 and the expiration of that turn-on time delay at 66.

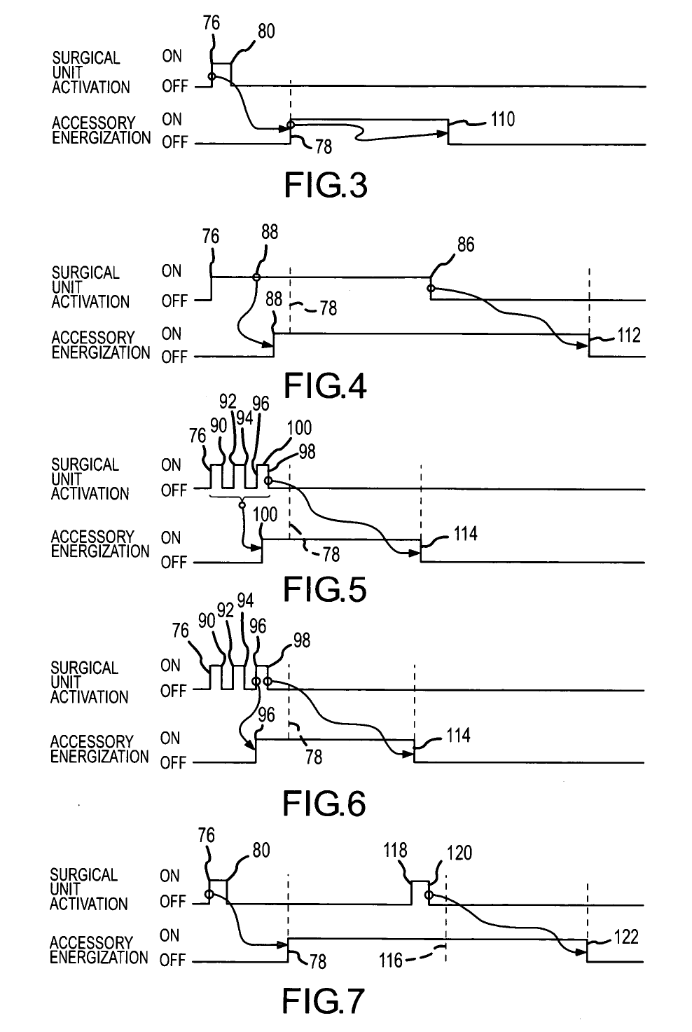

The functionality of energizing the supplemental accessory after the expiration of the basic turn-on time delay is graphically illustrated in FIG. 3. At time instant 76, the surgical unit asserts the activation signal (44, FIG. 1) in conjunction with delivering energy to the tissue of the patient. The activation signal sets the basic turn-on time delay (64, FIG. 2). The basic turn-on time delay expires (66, FIG. 2) at time instant 78. At time instant 78, the smoke evacuator or other supplemental accessory is energized as a result of closing the accessory switch at 68 (FIG. 2). Thus, the activation of the surgical unit at time instant 76 results in energizing the supplemental accessory at the time instant 78. The time between the time instants 76 and 78 is the amount of the basic turn-on time delay set at 66 (FIG. 2).

Under the program flow represented at 62, 64, 66 and 68 in FIG. 2 and shown at time instants 76 and 78 shown in FIG. 3, the basic turn on time delay, set at 64 (FIG. 2) controls the turn-on time delay in energizing the supplemental accessory at time instant 78 relative to the activation of the surgical unit at time instant 76. Typically, allowing the basic turn-on time delay to control energizing the supplemental accessory would normally occur in response to a single relatively short activation of the surgical unit to deliver a relatively short of burst of energy to the tissue. This circumstance is shown in FIG. 3, by the relatively short time between the activation time instant 76 and a deactivation time instant 80. The deactivation time instant 80 represents the time at which the electrosurgical generator 22 or other surgical unit is deactivated in response to the deassertion of the activation request signal 42 or 44 by opening the finger switch 36 or foot switch 38 (FIG. 1). Delivering a relatively short burst of energy might occur under circumstances where the surgeon is coagulating spot bleeders at a relatively slow pace, or where the surgeon is testing the application of energy to the tissue and the response of the tissue to the application of energy and prefers to perceive and sense the effects created without the disturbance of the noise and airflow from the activated smoke evacuator.

The control algorithm 60 shown in FIG. 2 evaluates two other conditions which have the potential for shortening the amount of the basic turn-on time delay established at 64. These other conditions offer the possibility of shortening the amount of time delay before the smoke evacuator or supplemental accessory is energized in response to activation of the surgical unit. As shown in FIG. 2, after the basic turn-on time delay has been set at 64 and the negative determination at 66 indicates that the basic turn-on time delay has not expired, a measurement is performed at 82 of the amount of time during which the surgical unit has been activated after the basic turn-on time delay was set at 64. A determination is next made at 84 as to whether the amount of time measured at 82 exceeds a predetermined percent of the basic turn-on time delay set at 64. For example, under circumstances where the surgical unit has been activated continuously for 50 to 75 percent of the turn-on time delay, it is preferable to activate the supplemental accessory before allowing the basic turn-on time delay to expire. Continuous activation offers the potential to generate the smoke, odor and particulate contaminants more quickly than under circumstances where a single burst of energy was delivered during the basic turn-on time delay. An affirmative determination at 84 causes the accessory switch to close at 68. Until the predetermined percentage of the turn-on time delay is reached, represented by a negative determination at 84, a looping evaluation of the time measurement at 82 continues until it is determined, at 84 that the time measurement at 82 constitutes the selected percent of the basic turn-on time delay. Preferably, the selected percent of the basic turn-on time delay is in the range of 50 to 75 percent of the amount of the basic turn-on time delay.

One example of circumstances where the functions performed at 82 and 84 shorten the basic turn-on time delay set at 64 is illustrated graphically in FIG. 4. The surgical unit has been activated at time instant 76, and the surgical unit remains activated until time instant 86. Thus, the surgical unit was activated continuously from time instant 76 throughout the time represented by the basic turn-on time delay at time instant 78. However, at a predetermined percentage of the turn-on time delay represented at time instant 88, for example in the range of 50 to 75 percent of the basic turn-on time delay, the measured amount of activation time (82, FIG. 2) reached the predetermined percentage of the basic time delay (determined at 84, FIG. 2), causing the accessory switch to close (68, FIG. 2) and energize the supplemental accessory. The supplemental accessory is shown in FIG. 4 as becoming energized at time instant 88. The time instant 88 occurs before the expiration of the basic turn-on time delay as shown at time instant 78.

Another example of the circumstances where the functions performed at 82 and 84 in the control algorithm 60 shorten the basic turn on time delay is illustrated graphically in FIG. 5. In the situation shown in FIG. 5, the surgical unit is being rapidly and intermittently activated. The first activation occurs at time instant 76, followed by a relatively quickly thereafter-occurring deactivation at time instant 90. Another activation quickly occurs at time instant 92, followed by another quick deactivation at time instant 94. A third activation occurs at time instant 96 followed by a quick deactivation at time instant 98. In essence, the surgical unit is activated and deactivated three times in relatively rapid succession during the basic turn-on time delay represented by the time instant 78. During each of the activations, the amount of time between time instances 76 and 90, 92 and 94, and 96 and 98 is measured at 82 (FIG. 2). The measured amount of time accumulated between the time instants 76 and 90, and between the time instants 92 and 94, and from the time instant 96 to the time instant 100 reaches a value where the accumulated time is equal to the predetermined percent of the basic turn on time delay as determined at 84 (FIG. 2), thereby causing the supplemental accessory to be energized at time instant 100. The time instant 100 occurs before the expiration of the basic turn-on time delay as shown at time instant 78.

The example shown in FIG. 5 occurs as a result of the total accumulated activation time exceeding the predetermined percentage of the basic turn-on time delay established at 84 (FIG. 2). If the rapid, intermittent activations are shorter in time duration, or less frequent in occurrence, the basic turn-on time delay would expire at time instant 78 (at 66, FIG. 2), before the total accumulated time of the activations reached the predetermined percent of the basic turn-on time delay. Under these circumstances, the basic turn-on time delay would control energization of the supplemental accessory.

The basic turn-on time delay can also be shortened from the basic turn-on time delay established at 64 by executing the process flow functions 102 and 104, shown in FIG. 2. So long as the basic turn-on time delay established at 64 has not expired as determined at 66, the number of activations of the surgical unit is counted at 102. If the number of activations counted at 102 is determined to reach a predetermined plurality of activations, for example three activations, an affirmative determination at 104 causes the accessory switch to close at 68. Until the number of activations counted at 102 reaches the predetermined plurality of activations established at 104, a wait loop is established at 104. The functionality represented at 102 and 104 activates the supplemental accessory under circumstances similar to those involved in rapidly and intermittently activating the surgical unit, except that the functionality represented at 102 and 104 is based solely on counting the number of activations, rather than counting the total time duration of the activations as represented by the functionality at 82 and 84.

An example of activating the supplemental accessory in response to a predetermined number of activations of the surgical unit is illustrated graphically in FIG. 6. In the example shown in FIG. 6, the number of activations which provides an affirmative determination at 104 (FIG. 2) is three. As shown in FIG. 6, the first activation begins at time instant 76 and ends at time instant 90. The second activation begins at time instant 92 and ends at time instant 94. The third activation begins at time instant 96 and ends at time instant 98. The third activation at time instant 96 is determined at 104 (FIG. 2) as affirmatively satisfying the count number requirement set at 102, causing the supplemental accessory to be energized at time instant 96. Energizing the supplemental accessory at time instant 96 occurs prior to expiration of the basic turn-on time delay at time instant 78 (established at 64, FIG. 2). The number of activations indicates the need to energize the smoke evacuator or supplemental accessory at an earlier time. The time instant 96 occurs before the expiration of the basic turn-on time delay as shown at time instant 78.

The number of the activations established at 104 is the basis for closing the accessory switch at 68, shown in FIG. 2, and must be established in relation to the length of the basic turn-on time delay and the quickness with which either the finger switch 36 or the foot switch 38 can be closed and opened by the surgeon. If the number of activations established to achieve an affirmative determination at 104 is too high to be achieved physically by the surgeon, the basic turn-on time delay established at 64 will expire at 66 before an affirmative determination is made at 104.

In summary, the control algorithm 60 energizes the supplemental accessory after a basic turn-on time delay as expired, or after a shortened turn-on time delay has expired. The situation where the turn-on time delay set at 64 (FIG. 2), has expired after the electrosurgical generator or surgical unit has been activated is exemplified by FIG. 3. FIG. 3 shows that the basic turn-on time delay governs when the supplemental accessory is energized, because no further activations of the surgical unit occur which might have the effect of shortening the basic turn-on time delay. The basic turn-on time delay is reduced under the conditions of an activation continuing for a predetermined percent of time of the basic turn-on time delay, as determined at 84 (FIG. 2), either as a result of continuous activation during the basic turn-on time delay, as exemplified by FIG. 4, or as a result of the series of activations which cumulatively reach the predetermined percent of the basic turn on time delay, as exemplified by FIG. 5. Additionally and alternatively, the basic turn-on time delay may be reduced under the condition of activating the electrosurgical generator or surgical unit a predetermined multiplicity of times during the basic turn-on time delay, as determined at 104 (FIG. 2), and as is exemplified in FIG. 6.

The control algorithm 60 also controls deenergizing the supplemental accessory after it has been energized following the turn-on time delay. Energizing the supplemental accessory occurs as a result of closing the accessory switch, as shown at 68 in FIG. 2. The control algorithm 60 maintains the supplemental accessory energized for a turn-off time delay, after the supplemental accessory has been energized. The turn-off time delay will be a predetermined turn-off time delay, for example two seconds, after the supplemental accessory was first energized or alternatively, after the supplemental accessory was last deenergized. As a result, the supplemental accessory will remain energized for the minimum duration of the predetermined turn-off time delay. Typically, the supplemental accessory will remain energized for as long as the surgical unit is activated plus the time duration of the predetermined turn-on time delay.

The functionality of the control algorithm 60 in controlling the deenergization of the supplemental accessory begins after the accessory switch has been closed at 68, as shown in FIG. 2, when the supplemental accessory becomes energized. A determination is made at 106 as to whether the surgical unit is activated after the accessory switch is closed at 68. The determination at 106 recognizes, immediately after the accessory switch is closed at 68, whether the surgical unit is activated. Because of the turn-on time delay which must elapse before the accessory switch is closed at 68, it is possible that the surgical unit will no longer be activated at the time that the accessory switch is closed at 68 and the supplemental accessory is energized. This situation is illustrated in FIG. 3, where the activation of the surgical unit begins at time instant 76 and terminates at time instant 80 before the supplemental accessory is energized at time instant 78 after the expiration of the basic turn-on time delay. Under the circumstances the negative determination at 106 shown in FIG. 2 sets a predetermined accessory turn-off time delay at 70. The predetermined turn-off time delay set at 70 will then control the time when the supplemental accessory will become deenergized as determined at 72, provided that the supplemental accessory is not activated again within the time duration while the supplemental accessory is energized, as determined at 108. So long as the surgical unit is not activated as determined at 108, a loop between the program flow functions 72 and 108 as established, until such time as the determination at 72 is affirmative and the accessory switch is opened at 74. An example of this situation is illustrated in FIG. 3, where the supplemental accessory is energized at time instant 78 and the expiration of the turn-off time delay at time instant 110 closes the accessory switch to deenergize the supplemental accessory. The time between the time instants 78 and 110 represents the amount of the turn-off delay set at 70 (FIG. 2).

On the other hand as shown in FIG. 2, if the surgical unit remains activated when the accessory switch 68 is closed, the program flow enters a wait loop at 106 until the surgical unit is no longer activated. A negative determination at 106 occurs when the surgical unit is deactivated. Thereafter, the turn-off time delay is set at 70. The determination at 72 is negative immediately after the turn-off time delay has been set at 70, because the turn-off time delay has not expired. Provided that the surgical unit is not activated, the determination at 108 will be negative, causing a program flow loop between 72 and 108 until the turn-off time delay has expired as determined at 72. At that point, the accessory switch is opened at 74. This situation is illustrated in FIG. 4. The activation of the surgical unit continues after the supplemental accessory has been energized until time instant 86. At time instant 80, negative determination at 106 (FIG. 2) results in setting the turn-off time delay at 70 (FIG. 2). Because the surgical unit is not thereafter activated as shown in FIG. 4, the turn-off time delay expires, and the accessory switch is opened to deenergize the supplemental accessory at the time instant 112. The time between the time instants 80 and 112 represents the amount of the turn-off time delay established at 70 (FIG. 2).

A similar situation is also represented in FIG. 5. At time instant 100, the accessory switch is closed and the supplemental accessory is energized. A short time thereafter at time instant 98, the surgical unit is deactivated, and is not thereafter activated. The deactivation of the surgical unit at time instant 98 results in the negative determination at 106 (FIG. 2) and the turn-off time delay is set at 70 (FIG. 2). Negative determinations occur at 106 and 108 (FIG. 2) in a continuous loop until the turn-off time delay has expired and the accessory switch is opened at time instant 114. The time duration between the time instants 98 and 114 shown in FIG. 5 represents the amount of the turn-off time delay set at 70 (FIG. 2).

Activating the surgical unit during the time that the supplemental accessory is energized resets or reestablishes the turn-off time delay, as understood from FIG. 2. After the accessory turn-off time delay has been set at 70, the determination at 72 will be negative for so long as the turn-off time delay has not expired. Each negative determination at 72 results in determining at 108 whether the surgical unit has become activated. Should the surgical unit become activated during the duration of the accessory turn-off time delay, as determined affirmatively at 108, the affirmative determination results in immediately redirecting the program flow to reset or reestablish the turn-off time delay at 70. This situation is illustrated in FIG. 7. The initial turn-on time delay is set at time instant 76 and would otherwise expire at time instant 116 as a result of setting that turn-off time delay at 70 (FIG. 2). The surgical unit is activated at time instant 118, before the expiration of the initially-set turn-off time delay at time instant 116. The surgical unit remains activated from time instant 118 to time instant 120. As understood from FIG. 2, the activation at time instant 118 is recognized by an affirmative determination at 108, resulting in resetting the turn-off time delay at 70. So long as the surgical unit remains activated between time instants 118 and 120 (FIG. 7), the affirmative determination at 108 continually resets or reestablishes the turn-off time delay at 70. At the time instant 120 (FIG. 7) when the surgical unit is no longer activated, the negative determination at 108 initiates a program flow loop between the negative determinations 72 and 108, until expiration of the most recent turn-off time delay (set at time instant 120, FIG. 7). The expiration of the most recently established turn-off time delay occurs at time instant 122, shown in FIG. 7. The time duration between the time instants as 120 and 122 is the amount of the turn-off time delay set at 104.

In summary, the control algorithm 60 deenergizes the smoke evacuator or other supplemental accessory after the expiration of a turn-off time delay, set at 70 (FIG. 2). The supplemental accessory remains energized during the duration of the turn-off time delay. The turn-off time delay commences beginning with energizing the supplemental accessory or after the surgical unit was last deactivated, which ever results in the supplemental accessory remaining energized longer. Should the surgical unit be activated during the time that the supplemental accessory is energized, the supplemental accessory remains energized until the turn-off time delay has expired measured from the deactivation of the surgical unit.

The functionality of the control algorithm achieves significant improvements. The short turn-on time delay allows the surgeon to sense and observe aspects of the surgical procedure without the sensory distraction of immediately energizing the smoke evacuator or other supplemental accessory. The surgeon has an opportunity to sense the initial effect of the energy on the tissue, to gauge the amount of energy delivered and to evaluate the response of the tissue to that energy, without the potential distraction of sensing these factors because the added noise and air movement at the surgical site created by immediately energizing the smoke evacuator or supplemental accessory. The short turn-on time delay does not permit an excessive accumulation of smoke, odor and particulates to the point where removal of those contaminants by the smoke evacuator is impossible or difficult after the short turn-on time delay. On the other hand, continuous activation or rapid intermittent activation of the surgical unit during the basic turn-on time delay indicates that shortening the turn-on time delay may be desirable. Activation of the surgical unit continuously during the initial turn-on delay time, or for a predetermined significant portion of the initial turn-on time delay, or based on a predetermined plurality of rapid intermittent activations during the initial turn-on time delay, shortens the turn-on time delay to allow the supplemental accessory to be energized somewhat earlier. Deenergizing the supplemental accessory after the predetermined turn-off time delay minimizes the number of stops and starts of the smoke evacuator or other supplemental accessory, thereby reducing the added wear created by repeatedly stopping and starting the supplemental accessory. Resetting or reestablishing the turn-off delay time by activating the surgical unit while the supplemental accessory is energized continues the continuous evacuation of smoke, odor and particulate contaminants from the surgical site or the other functions of the supplemental accessory during the progress of the surgical procedure. Many other advantages and improvements will be apparent upon gaining a complete understanding and appreciation of the present invention.

A presently preferred embodiment of the present invention and many of its improvements have been described with a degree of particularity. This description is a preferred example of implementing the invention, and is not necessarily intended to limit the scope of the invention. The scope of the invention is defined by the following claims.

The inventions claimed:

1. A method of energizing a supplemental accessory in relation to activating a surgical unit to apply energy, comprising:
activating the surgical unit to apply energy; and
energizing the supplemental accessory after expiration of a predetermined turn-on time delay after the surgical unit has been activated.

2. A method as defined in claim 1, further comprising:
establishing the predetermined turn-on time delay as a basic fixed amount of time.

3. A method as defined in claim 2, further comprising:
establishing the predetermined turn-on time delay as an amount of time less than the basic fixed amount of time upon activating the surgical unit for a predetermined percentage of the basic fixed amount of time.

4. A method as defined in claim 3, further comprising:
establishing the predetermined percentage within the range of 50-75 percent of the basic fixed amount of time.

5. A method as defined in claim 4, further comprising:
establishing the basic fixed amount of time at approximately one second.

6. A method as defined in claim 3, further comprising:
activating the surgical unit multiple times during the basic fixed amount of time;
counting the amount of time that the surgical unit is activated during each one of the multiple activations during the fixed amount of time; and
energizing the supplemental accessory when the cumulative amount of time of the multiple activations during the basic fixed amount of time equals the predetermined percentage of the fixed amount of time.

7. A method as defined in claim 3, further comprising:
continuously activating the surgical unit during the basic fixed amount of time;
counting the amount of time that the surgical unit is continuously activated during the basic fixed amount of time; and
energizing the supplemental accessory when the cumulative amount of time of the continuous activation equals the predetermined percentage of the fixed amount of time.

8. A method as defined in claim 3, further comprising:
activating the surgical unit multiple times during the basic fixed amount of time;
counting the number of multiple activations of the surgical unit during the basic fixed amount of time; and
energizing the supplemental accessory when the counted number of activations equals a predetermined count number greater than one.

9. A method as defined in claim 2, further comprising:
activating the surgical unit multiple times during the basic fixed amount of time;
counting the number of multiple activations of the surgical unit during the basic fixed amount of time; and
energizing the supplemental accessory when the counted number of activations equals a predetermined count number greater than one.

10. A method as defined in claim 1, further comprising:
deactivating the surgical unit to cease applying energy after the surgical unit has been activated; and
deenergizing the supplemental accessory after the expiration of a predetermined turn-off time delay after the surgical unit has been deactivated.

11. A method as defined in claim 10, further comprising:
establishing the predetermined turn-off time delay as a greater amount of time than the predetermined turn-on time delay.

12. A method as defined in claim 10, further comprising:
establishing the basic fixed amount of time at approximately one second; and
establishing the predetermined turn-off time delay at approximately two seconds.

13. A method as defined in claim 10, further comprising:
deactivating the surgical unit during the predetermined turn-on time delay before the supplemental accessory is energized; and
deenergizing the supplemental accessory after the expiration of the predetermined turn-off time delay after the supplemental accessory has been energized.

14. A method as defined in claim 10, further comprising:
deactivating the surgical unit after expiration of the predetermined turn-on time delay while the supplemental accessory is energized; and
deenergizing the supplemental accessory after the expiration of the predetermined turn-off time delay from the time that the surgical unit was deactivated.

15. A method as defined in claim 10, further comprising:
activating the surgical unit while the supplemental accessory is energized;
deactivating the surgical unit while the supplemental accessory is energized; and
deenergizing the supplemental accessory after the expiration of the predetermined turn-off time delay from the time that the surgical unit was last deactivated when the supplemental accessory was energized.

16. A method as defined in claim 1, further comprising:
selecting the surgical unit as one of an electrosurgical generator or a medical laser; and
selecting the supplemental accessory as one of a smoke evacuator, lavage pump, or fluid pump.

17. A method as defined in claim 1, further comprising:
establishing the predetermined turn-on time delay as the earliest to occur one of a basic fixed amount of time elapsing after the surgical unit has been activated, or a predetermined percentage less than all of the basic fixed amount of time measured after the surgical unit has been activated, or a predetermined multiple number of activations of the surgical unit during the basic fixed amount of time measured after the surgical unit has been activated.

18. A method as defined in claim 17, further comprising:
deactivating the surgical unit to cease applying energy; and
deenergizing the supplemental accessory upon expiration of a predetermined turn-off time delay after the surgical unit was last deactivated while the supplemental accessory was energized or upon expiration of the predetermined turn-off time delay after the supplemental accessory was energized when the surgical unit was deactivated prior to energizing the supplemental accessory.

19. A method as defined in claim 18, or in the surgical unit is an electrosurgical generator and the supplemental accessory is a smoke evacuator.

20. A controller for energizing a supplemental accessory in relation to activation of a surgical unit to apply energy, the controller executing a control algorithm in response to an activation signal asserted by the surgical unit upon delivering energy, the control algorithm operatively closing an accessory switch to energize the supplemental accessory and opening the accessory switch to deenergize the supplemental accessory; the control algorithm operatively causing the controller to:
close the accessory switch after expiration of a predetermined turn-on time delay after the assertion of the activation signal.

21. A controller as defined in claim 20, wherein:
the predetermined turn-on time delay is a basic fixed amount of time.

22. A controller as defined in claim 21, wherein the control algorithm operatively causes the controller to:
count the amount of time that the activation signal is asserted during the basic fixed amount of time; and
close the accessory switch before expiration of the basic fixed amount of time when the counted amount of time that the activation signal is asserted equals a predetermined percentage less than all of the basic fixed amount of time.

23. A controller as defined in claim 21, wherein the control algorithm operatively causes the controller to:
count the number of assertions of the activation signal during the basic fixed amount of time; and
close the accessory switch before expiration of the basic fixed amount of time upon the counted number of assertions of the activation signal equaling a predetermined number greater than one.

24. A controller as defined in claim 21, wherein the control algorithm operatively causes the controller to:
establish the predetermined turn-on time delay as the earliest one to occur of the expiration of the basic fixed amount of time after assertion of the activation signal, of the activation signal being asserted for a predetermined percentage less than all of the basic fixed amount of time, or of the activation signal being asserted a predetermined multiple number of times during the basic fixed amount of time.

25. A controller as defined in claim 21, wherein the control algorithm operatively causes the controller to:
open the accessory switch after the expiration of a predetermined turn-off time delay after the last to occur of the deassertion of the activation signal or closure of the accessory switch.

26. A controller as defined in claim 21, wherein the surgical unit is an electrosurgical generator and the supplemental accessory is a smoke evacuator.
